(19) United States
(12) Patent Application Publication
(10) Pub. No.: US 2008/0108868 A1
(43) Pub. Date: May 8, 2008

(54) Device, System and Method of In-Vivo Electro-Stimulation

(76) Inventors: Christopher Paul Swain, London (GB); Charles Alexander Mosse, London (GB); Timothy Noel Mills, London (GB)

Correspondence Address: Pearl Cohen Zedek Latzer, LLP, 1500 Broadway, 12th Floor, New York, NY, 10036, US

(21) Appl. No.: 11/792,254

(22) PCT Filed: Dec. 4, 2005
(86) PCT No.: PCT/IL05/01299

§ 371 Date: Jun. 4, 2007

Related U.S. Application Data
(60) Provisional application No. 60/632,260, filed on Dec. 2, 2004.

(51) Int. Cl. A61B 1/00

(52) U.S. Cl. 600/104

(57) ABSTRACT

An autonomous in-vivo device (140) includes: an in-vivo electro-stimulation unit (190, 192) to electro-stimulate at least a portion of a body lumen; and an imager (146) to acquire in-vivo images.

FIELD OF THE INVENTION

The present invention relates to the field of in-vivo operations. More specifically, the present invention relates to devices, systems and methods for performing in-vivo electro-stimulation.

### BACKGROUND OF THE INVENTION

Some in-vivo sensing systems may include an in-vivo imaging device able to acquire and transmit images of, for example, the gastrointestinal GI tract while the in-vivo imaging device passes through the GI lumen.

Other devices, systems and methods for in-vivo sensing of passages or cavities within a body, and for sensing and gathering information (e.g., image information, pH information, temperature information, electrical impedance information, pressure information, etc.), are known in the art.

### SUMMARY OF THE INVENTION

Some embodiments of the present invention may include, for example, devices, systems and methods for in-vivo electro-stimulation.

In some embodiments, for example, an in-vivo electro-stimulation device may be autonomous. In some embodiments, for example, the in-vivo electro-stimulation device may be swallowable, e.g., a swallowable capsule which may be swallowed and moved through the GI tract.

In some embodiments, for example, the in-vivo device may include an in-vivo electro-stimulation unit to electro-stimulate at least a portion of a body lumen; and an imager to acquire in-vivo images.

In some embodiments, for example, the in-vivo device may include a receiver to receive a signal, typically from a remote or external source, representing an in-vivo electro-stimulation command and/or parameter. According to some embodiments radio frequency is used to remotely control electrostimulation.

In some embodiments, for example, the in-vivo device may include a power source to selectively provide power to the in-vivo electro-stimulation unit based on a received signal, or based on a pre-programmed in-vivo electro-stimulation scheme.

In some embodiments, for example, the in-vivo electro-stimulation unit is located at a location of the autonomous in-vivo device, and wherein the location is substantially non-obscuring relative to a field-of-view imaged by the imager.

In some embodiments, for example, the in-vivo device may include a visual indicator operatively coupled to the in-vivo electro-stimulation unit, wherein the visual indicator provides a visual indication upon in-vivo electro-stimulation.

In some embodiments, for example, the in-vivo device may include a front in-vivo electro-stimulation unit located at a front side of the autonomous in-vivo device; and a back in-vivo electro-stimulation unit located at a back side of the autonomous in-vivo device. In some embodiments, for example, the in-vivo device may include a front visual indicator operatively coupled to the front in-vivo electro-stimulation unit; and a back visual indicator operatively coupled to the back in-vivo electro-stimulation unit. In some embodiments, for example, the front visual indicator is to generate a visual indication having a first color, and the back visual indicator is to generate a visual indication having a second, different, color.

In some embodiments, for example, a system may include an autonomous in-vivo imaging device including at least an imager to acquire in-vivo images; and an autonomous in-vivo electro-stimulation device including at least an electro-stimulation unit.

In some embodiments, for example, the imager is to acquire an in-vivo image including at least a portion, or a visual indicator, of the in-vivo electro-stimulation device.

In some embodiments, for example, the system may include a connector to connect the in-vivo imaging device and the in-vivo electro-stimulation device.

In some embodiments, for example, a method may include receiving a signal representing an in-vivo electro-stimulation command; and based on the in-vivo electro-stimulation command, operating an in-vivo electro-stimulation unit.

In some embodiments, for example, the method may include receiving a signal representing an in-vivo electro-stimulation parameter; and based on the in-vivo electro-stimulation parameter, operating the in-vivo electro-stimulation unit.

In some embodiments, for example, the method may include selectively activating and/or deactivating the in-vivo electro-stimulation unit.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic illustration of an in-vivo system according to an embodiment of the invention;

FIG. 2 is a schematic illustration of a set of multiple in-vivo devices according to an embodiment of the invention; and

FIG. 3 is a flow-chart of a method of in-vivo electro-stimulation according to an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements throughout the serial views.

### DETAILED DESCRIPTION OF THE INVENTION

In the following detailed description, numerous specific details are set forth in order to provide a thorough understanding of the present invention. However, it will be understood by those skilled in the art that the present invention may be practiced without these specific details. In other instances, well-known methods, procedures, and components have not been described in detail so as not to obscure the present invention. Some embodiments of the present invention are directed to a typically swallowable in-vivo device, e.g., a typically swallowable in-vivo sensing or imaging device. Devices according to embodiments of the present invention may be similar to embodiments described in U.S. patent application Ser. No. 09/800,470, entitled “Device and System for In-vivo Imaging”, filed on 8 Mar. 2001, published on Nov. 1, 2001 as United States Patent Application Publication Number 2001/0035902, and/or in U.S. Pat. No. 5,604,531 to Iddan et al., entitled “In-Vivo Video Camera System”, and/or in U.S. patent application Ser. No. 10/046,541, filed on Jan. 16, 2002, published on Aug. 15, 2002 as United States Patent Application Publication Number 2002/0109774, all of which are hereby incorporated by reference. An external receiver/recorder unit, a processor and a monitor, e.g., in a workstation, such as those described in the above publications, may be suitable for use with some embodiments of the present invention. Devices and systems as described herein may have other configurations and/or other sets of components. For example, the present invention may be practiced using an endoscope, needle, stent, catheter, etc. Some in-vivo devices may be capsule shaped, or may have other shapes, for example, a peanut shape or tubular, spherical, conical, or other suitable shapes.

Some embodiments of the present invention may include, for example, a typically swallowable in-vivo device. In other embodiments, an in-vivo device need not be swallowable and/or autonomous, and may have other shapes or configurations. Some embodiments may be used in various body lumens, for example, the GI tract, blood vessels, the urinary tract, the reproductive tract, or the like. In some embodiments, the in-vivo device may optionally include a sensor, an imager, and/or other suitable components.

Embodiments of the in-vivo device are typically autonomous and are typically self-contained. For example, the in-vivo device may be or may include a capsule or other unit where all the components are substantially contained within a container, housing or shell, and where the in-vivo device does not require any wires or cables to, for example, receive power or transmit information. The in-vivo device may communicate with an external receiving and display system to provide display of data, control, or other functions. For example, power may be provided by an internal battery or a wireless receiving system. Other embodiments may have other configurations and capabilities. For example, components may be distributed over multiple sites or units. Control information may be received from an external source.

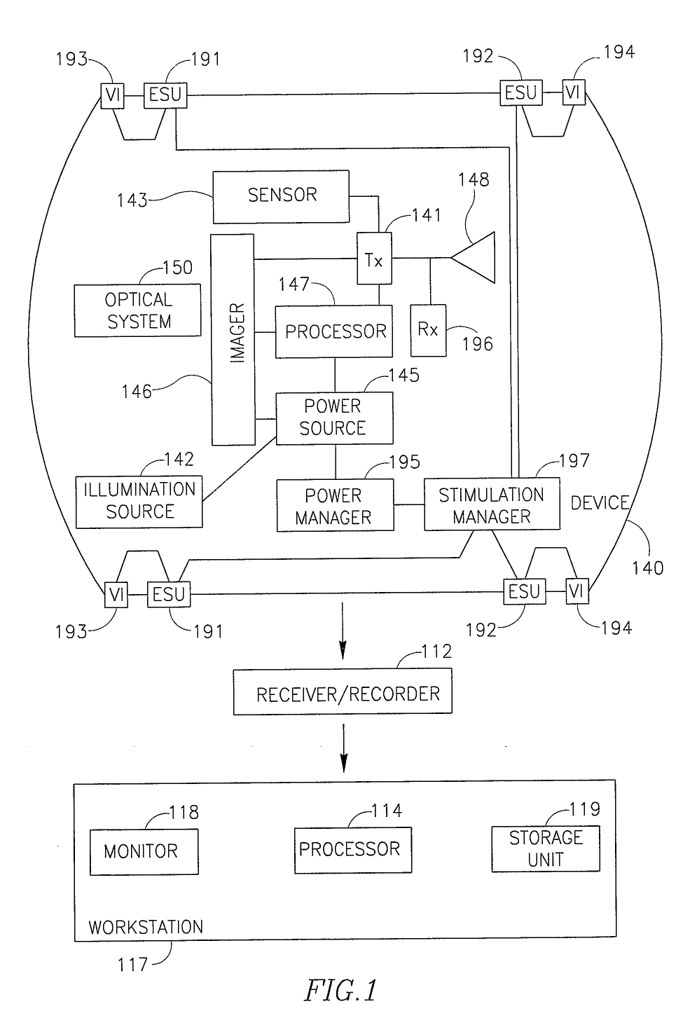

FIG. 1 schematically illustrates an in-vivo system in accordance with some embodiments of the present invention. One or more components of the system may be used in conjunction with, or may be operatively associated with, the devices and/or components described herein or other in-vivo devices in accordance with embodiments of the invention.

In some embodiments, the system may include a device 140 having a sensor, e.g., an imager 146, one or more illumination sources 142, a power source 145, and a transmitter 141. In some embodiments, device 140 may be implemented using a swallowable capsule, but other sorts of devices or suitable implementations may be used. Outside a patient's body may be, for example, an external receiver/recorder 112 (including, or operatively associated with, for example, one or more antennas, or an antenna array), a storage unit 119, a processor 114, and a monitor 118. In some embodiments, for example, processor 114, storage unit 119 and/or monitor 118 may be implemented as a workstation 117, e.g., a computer or a computing platform.

Transmitter 141 may operate using radio waves; but in some embodiments, such as those where device 140 is or is included within an endoscope, transmitter 141 may transmit/receive data via, for example, wire, optical fiber and/or other suitable methods. Other known wireless methods of transmission may be used. Transmitter 141 may include, for example, a transmitter module or sub-unit and a receiver module or sub-unit, or an integrated transceiver or transmitter-receiver.

Device 140 typically may be or may include an autonomous swallowable capsule, but device 140 may have other shapes and need not be swallowable or autonomous. Embodiments of device 140 are typically autonomous, and are typically self-contained. For example, device 140 may be a capsule or other unit where all the components are substantially contained within a container or shell, and where device 140 does not require any wires or cables to, for example, receive power or transmit information. In some embodiments, device 140 may be autonomous and non-remote-controllable; in another embodiment, device 140 may be partially or entirely remote-controllable.

In some embodiments, device 140 may communicate with an external receiving and display system (e.g., workstation 117 or monitor 118) to provide display of data, control, or other functions. For example, power may be provided to device 140 using an internal battery, an internal power source, or a wireless system able to receive power. Other embodiments may have other configurations and capabilities. For example, components may be distributed over multiple sites or units, and control information or other information may be received from an external source.

In some embodiments, device 140 may include an in-vivo video camera, for example, imager 146, which may capture and transmit images of, for example, the GI tract while device 140 passes through the GI lumen. Other lumens and/or body cavities may be imaged and/or sensed by device 140. In some embodiments, imager 146 may include, for example, a Charge Coupled Device (CCD) camera or imager, a Complementary Metal Oxide Semiconductor (CMOS) camera or imager, a digital camera, a stills camera, a video camera, or other suitable imagers, cameras, or image acquisition components.

In some embodiments, imager 146 in device 140 may be operationally connected to transmitter 141. Transmitter 141 may transmit images to, for example, external transceiver or receiver/recorder 112 (e.g., through one or more antennas), which may send the data to processor 114 and/or to storage unit 119. Transmitter 141 may also include control capability, although control capability may be included in a separate component, e.g., processor 147. Transmitter 141 may include any suitable transmitter able to transmit image data, other sensed data, and/or other data (e.g., control data) to a receiving device. Transmitter 141 may also be capable of receiving signals/commands, for example from an external transceiver. For example, in some embodiments, transmitter 141 may include an ultra low power Radio Frequency (RF) high bandwidth transmitter, possibly provided in Chip Scale Package (CSP).

In some embodiment, transmitter 141 may transmit/receive via antenna 148. Transmitter 141 and/or another unit in device 140, e.g., a controller or processor 147, may include control capability, for example, one or more control modules, processing module, circuitry and/or functionality for controlling device 140, for controlling the operational mode or settings of device 140, and/or for performing control operations or processing operations within device 140. According to some embodiments, transmitter 141 may include a receiver which may receive signals (e.g., from outside the patient's body), for example, through antenna 148 or through a different antenna or receiving element. According to some embodiments, signals or data may be received by a separate receiving device in device 140.

Power source 145 may include one or more batteries or power cells. For example, power source 145 may include silver oxide batteries, lithium batteries, other suitable electrochemical cells having a high energy density, or the like. Other suitable power sources may be used. For example, power source 145 may receive power or energy from an external power source (e.g., an electromagnetic field generator), which may be used to transmit power or energy to in-vivo device 140.

In some embodiments, power source 145 may be internal to device 140, and/or may not require coupling to an external power source, e.g., to receive power. Power source 145 may provide power to one or more components of device 140 continuously, substantially continuously, or in a non-discrete manner or timing, or in a periodic manner, an intermittent manner, or an otherwise non-continuous manner. In some embodiments, power source 145 may provide power to one or more components of device 140, for example, not necessarily upon-demand, or not necessarily upon a triggering event or an external activation or external excitement.

Optionally, in some embodiments, transmitter 141 may include a processing unit or processor or controller, for example, to process signals and/or data generated by imager 146. In another embodiment, the processing unit may be implemented using a separate component within device 140, e.g., controller or processor 147, or may be implemented as an integral part of imager 146, transmitter 141, or another component, or may not be needed. The processing unit may include, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In some embodiments, for example, the processing unit or controller may be embedded in or integrated with transmitter 141, and may be implemented, for example, using an ASIC.

In some embodiments, imager 146 may acquire in-vivo images continuously, substantially continuously, or in a non-discrete manner, for example, not necessarily upon-demand, or not necessarily upon a triggering event or an external activation or external excitement; or in a periodic manner, an intermittent manner, or an otherwise non-continuous manner.

In some embodiments, transmitter 141 may transmit image data continuously, or substantially continuously, for example, not necessarily upon-demand, or not necessarily upon a triggering event or an external activation or external excitement; or in a periodic manner, an intermittent manner, or an otherwise non-continuous manner.

In some embodiments, device 140 may include one or more illumination sources 142, for example one or more Light Emitting Diodes (LEDs), “white LEDs”, or other suitable light sources. Illumination sources 142 may, for example, illuminate a body lumen or cavity being imaged and/or sensed. An optional optical system 150, including, for example, one or more optical elements, such as one or more lenses or composite lens assemblies, one or more suitable optical filters, or any other suitable optical elements, may optionally be included in device 140 and may aid in focusing reflected light onto imager 146, focusing illuminated light, and/or performing other light processing operations.

In some embodiments, illumination source(s) 142 may illuminate continuously, or substantially continuously, for example, not necessarily upon-demand, or not necessarily upon a triggering event or an external activation or external excitement. In some embodiments, for example, illumination source(s) 142 may illuminate a pre-defined number of times per second (e.g., two or four times), substantially continuously, e.g., for a time period of two hours, four hours, eight hours, or the like; or in a periodic manner, an intermittent manner, or an otherwise non-continuous manner.

In some embodiments, the components of device 140 may be enclosed within a housing or shell, e.g., capsule-shaped, oval, or having other suitable shapes. The housing or shell may be substantially transparent or semi-transparent, and/or may include one or more portions, windows or domes which may be substantially transparent or semi-transparent. For example, one or more illumination source(s) 142 within device 140 may illuminate a body lumen through a transparent or semi-transparent portion, window or dome; and light reflected from the body lumen may enter the device 140, for example, through the same transparent or semi-transparent portion, window or dome, or, optionally, through another transparent or semi-transparent portion, window or dome, and may be received by optical system 150 and/or imager 146. In some embodiments, for example, optical system 150 and/or imager 146 may receive light, reflected from a body lumen, through the same window or dome through which illumination source(s) 142 illuminate the body lumen.

Data processor 114 may analyze the data received via external receiver/recorder 112 from device 140, and may be in communication with storage unit 119, e.g., transferring frame data to and from storage unit 119. Data processor 114 may provide the analyzed data to monitor 118, where a user (e.g., a physician) may view or otherwise use the data. In some embodiments, data processor 114 may be configured for real time processing and/or for post processing to be performed and/or viewed at a later time. In the case that control capability (e.g., delay, timing, etc) is external to device 140, a suitable external device (such as, for example, data processor 114 or external receiver/recorder 112 having a transmitter or transceiver) may transmit one or more control signals to device 140.

Monitor 118 may include, for example, one or more screens, monitors, or suitable display units. Monitor 118, for example, may display one or more images or a stream of images captured and/or transmitted by device 140, e.g., images of the GI tract or of other imaged body lumen or cavity. Additionally or alternatively, monitor 118 may display, for example, control data, location or position data (e.g., data describing or indicating the location or the relative location of device 140), orientation data, and various other suitable data. In some embodiments, for example, both an image and its position (e.g., relative to the body lumen being imaged) or location may be presented using monitor 118 and/or may be stored using storage unit 119. Other systems and methods of storing and/or displaying collected image data and/or other data may be used.

Typically, device 140 may transmit image information in discrete portions. Each portion may typically correspond to an image or a frame; other suitable transmission methods may be used. For example, in some embodiments, device 140 may capture and/or acquire an image once every half second, and may transmit the image data to external receiver/recorder 112. Other constant and/or variable capture rates and/or transmission rates may be used.

Typically, the image data recorded and transmitted may include digital color image data; in alternate embodiments, other image formats (e.g., black and white image data) may be used. In some embodiments, each frame of image data may include 256 rows, each row may include 256 pixels, and each pixel may include data for color and brightness according to known methods. According to other embodiments a 320×320 pixel imager may be used. Pixel size may be between 5 to 6 micron. According to some embodiments pixels may be each fitted with a micro lens. For example, a Bayer color filter may be applied. Other suitable data formats may be used, and other suitable numbers or types of rows, columns, arrays, pixels, sub-pixels, boxes, super-pixels and/or colors may be used. Optionally, device 140 may include one or more sensors 143, instead of or in addition to a sensor such as imager 146. Sensor 143 may, for example, sense, detect, determine and/or measure one or more values of properties or characteristics of the surrounding of device 140. For example, sensor 143 may include a pH sensor, a temperature sensor, an electrical conductivity sensor, a pressure sensor, or any other known suitable in-vivo sensor.

In some embodiments, device 140 may include one or more Electro-Stimulation Units (ESUs), for example, one or more front ESU(s) 191 and/or one or more back ESU(s) 192. The ESU(s) 191-192 may include, for example, one or more electrodes, bi-polar electrodes, electric components, conductive elements, or other suitable components able to perform electro-stimulation. The ESU(s) 191-192 may be operatively connected to power source 145 to receive power for the electro-stimulation.

The ESU(s) 191-192 may be located, for example, near or at external portion(s) of device 140, e.g., close to the exterior of device 140, embedded within a housing (e.g., a capsule housing) which may enclose or encapsulate the components of device 140, or the like. ESU(s) 191-192 may, according to some embodiments be 5 mm in width and may be spaced 3 mm apart from each other around the circumference of a capsule shaped device. In some embodiments, the ESU(s) 191-192 may be internal and/or external to device 140. In some embodiments, the ESU(s) 191-192 may be located at one or more locations which may not interfere with, or otherwise obscure, a field-of-view which may be imaged by imager 146. For example, the ESU(s) 191-192 may be external or semi-external to device 146, may be located at a portion of device 140 which is not in the general direction of imaging of imager 146, may be located at a side portion of device 140, or the like.

In some embodiments, power (e.g., voltage or current) may be applied to one or more ESU(s) 191-192, for example, selectively, at pre-defined time intervals, substantially continuously, at one ore more pre-defined time(s), on demand, in response to a remote instruction, in response to an external instruction, or the like. Upon application of power, ESU(s) 191-192 may electro-stimulate a tissue, e.g., a muscle tissue, a muscle, a nerve, or a wall of a body lumen. Typically, in the GI tract circular muscles of the GI tract wall are stimulated. This may, for example, cause a tissue or a body lumen to contract, e.g., around or near the ESU(s) 191-192 that performed electro-stimulation. In some embodiments, the contraction may propel, advance, urge, accelerate, decelerate, push, pull, squeeze, or otherwise move the device 140, for example, forward, backward, or sideways, or may otherwise cause device 140 to change its position, location, orientation or direction. The electro-stimulation may optionally be repeated, e.g., to further move or advance the device 140 within the body lumen. In some embodiments, the contraction may propagate along the body lumen or the tissue, thereby further advancing the device 140. In some embodiments, electrodes fired in sequence might mimic or induce peristalsis (i.e. sequenced contraction). Optionally, the ESU(s) 191-192 may cease to perform electro-stimulation, for example, to decelerate the device 140.

In some embodiments, the ESU(s) 191-192 may be controlled remotely, e.g., in response to commands received by wireless signals from a transmitter external to the human body. For example, device 140 may include a wireless receiver 196, which may be implemented, for example, as a stand-alone unit, using an ASIC, as part of transmitter 141 (e.g., as a transceiver or transmitter-receiver unit), or the like. Receiver 196 may be operatively coupled to antenna 148, and may receive signals, e.g., from an external transmitter. An RF receiver may be used, which may be similar to receivers used in remote radio controlled toys. A remote transmitter, typically an RF transmitter, may be included in the external image receiving system. For example, external receiver/recorder 112 may include an RF or other suitable transmitter which may be controlled by an external operator to send commands and control electrostimulation within the device 140. Other frequencies and other transmitting techniques may be used, for example, bluetooth technology may be used to send signals in to device 140.

The received signals may represent, for example, an instruction to perform electro-stimulation, an instruction to cease electro-stimulation, a value of an electro-stimulation parameter which may be used by ESU(s) 191-192 (e.g., a voltage value, a current value, a power-level value, a time interval value, a value indicating a number of electric pulses, etc.), or other suitable data. The data may be processed, for example, using a stimulation manager 197, which may operate, activate and/or deactivate one or more of the ESU(s) 191-192 based on the received signals.

Optionally, one or more of the ESU(s) 191-192 may be operatively connected to a visual indicator. For example, visual indicator(s) 193 and 194 may be operatively coupled to ESU(s) 191 and 192, respectively. The visual indicator(s) 193-194 may include, for example, one or more LEDs or color LEDs, or other suitable illumination sources. In some embodiment, for example, various visual indicator(s) 193-194 may be able to illuminate in various, respective, colors; for example, visual indicator(s) 193 may be able to illuminate in green color, whereas visual indicator(s) 194 may be able to illuminate in yellow color. Other suitable colors may be used. In some embodiments, for example, the visual indicator(s) 193-194 may illuminate when electro-stimulation is performed by ESU(s) 191-192, and may not illuminate when electro-stimulation is not performed by ESU(s) 191-192. According to other embodiments different optical signals (e.g., different color lights) may indicate a different direction of movement of the device (e.g., forwards or backwards). This may allow, for example, a visual or optical indication that ESU(s) 191-192 function and/or perform electro-stimulation. Visual indications may also be useful in interventional procedures such as laparoscopy, in which an imaging capsule may be used.

In some embodiments, ESU(s) 191-192 may receive a voltage of, for example, between 2 Volts and 10 Volts, e.g., 3 Volts or 5 Volts; other suitable voltage values may be used. In some embodiments, ESU(s) 191-192 may receive voltage, for example, at a frequency of between 3 Hz to 20 KHz, or between 10 Hz to 30 Hz; other suitable frequency values may be used.

In some embodiments, electro-stimulation by front ESU(s) 191 may move the device 140 backwards, whereas electro-stimulation by back ESU(s) 192 may move the device 140 forward, or vice versa. ESU(s) 191-192 may be located at other portions of device 140, and may be used to result in other types of movement of device 140. In some embodiments, electro-stimulation of some or all of the ESU(s) 191-192 may substantially hold or “lock” device 140 to its location. For example, stimulation applied simultaneously to both ends of an egg or capsule shaped device, typically when both ends of the device are in contact with tissue, may cause the device to stop in one location. Other suitable movement schemes may be used, e.g., by selectively activating and/or de-activating one or more of ESU(s) 191-192.

In some embodiments, device 140 may optionally include a power manager 195, e.g., to control the power provided by power source 145 to ESU(s) 191-192. This may be performed, for example, based on an activation scheme operated by stimulation manager 197, and/or based on external signals or commands (e.g., received by wireless receiver 196). In some embodiments, optionally, power manager 195 may selectively provide power to various components of device 140; for example, imager 146 may not image when ESU(s) 191-192 are activated, or vice versa.

In some embodiments, stimulation manager 197 need not be operatively connected to receiver 196, and/or need not operate based on external signals. For example, stimulation manager may utilize a pre-programmed or pre-configured stimulation scheme, e.g., including the timing or intervals of electro-stimulation pulses, the power level(s) used for electro-stimulation, or other parameters.

Additionally or alternatively, stimulation manager 197 may optionally activate one or more ESU(s) 191-192 upon occurrence of a triggering event, for example, when device 140 does not move for a pre-defined period of time, when device 140 moves at a relatively slow speed (e.g., smaller than a threshold value), when device 140 reaches a certain in-vivo location or condition, when sensor 143 senses certain information (e.g., temperature information, pH information, pressure information, or the like), or other triggering events. In some embodiments, stimulation manager 197 may selectively activate one or more ESU(s) 191-192 based on localization data of device 140, based on analysis of one or more images captured by imager 146, in response to detection of an abnormality or a pathology, or the like.

In some embodiments, optionally, device 140 may be substantially egg-shaped or oval-shaped. This may, for example, allow device 140 to have improved responsiveness to body lumen contractions resulting from the electro-stimulation, and may further facilitate the movement of device 140 in-vivo. Other suitable shapes may be used.

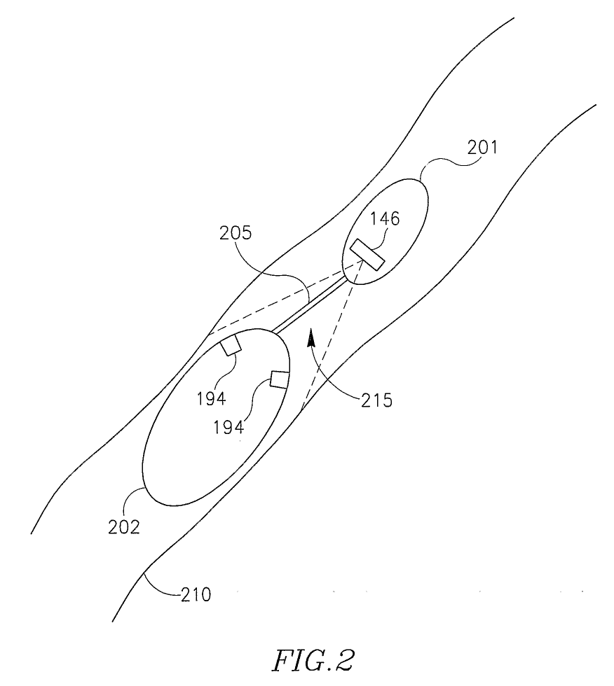

FIG. 2 schematically illustrates a set of multiple in-vivo devices in accordance with an embodiment of the invention. An in-vivo imaging device 201 and an in-vivo electro-stimulation device 202 may be inserted into a patient's body, e.g., one after the other or substantially simultaneously, and may pass through a body lumen 210. The in-vivo imaging device 201 may acquire images in-vivo, whereas the in-vivo electro-stimulation device 202 may perform electro-stimulation in-vivo. For example, the imaging-related components of device 140 of FIG. 1 may be included in the in-vivo imaging device 201, whereas the electro-stimulation-related components of device 140 of FIG. 1 may be included in the in-vivo electro-stimulation device 202.

Optionally, in-vivo imaging device 201 may acquire images which may include the in-vivo electro-stimulation device 202 or a portion thereof. For example, the in-vivo imaging device 201 may acquire images showing one or more visual indicator(s) 194 of the in-vivo electro-stimulation device 202. This may allow, for example, a visual identification or analysis of the operation of the in-vivo electro-stimulation device 202, e.g., based on the images captured by the in-vivo imaging device 201. Optionally, the focus, the field-of-view or other optical parameters of imager 146 may be configured or adapted such that in-vivo electro-stimulation device 202, or a portion thereof, may be in a field-of-view 215 which may be imaged by imager 146 of in-vivo imaging device 201.

In some embodiments, optionally, a spacer, a thread, a coaxial cable, a cable, or other connector 205 may connect or join the multiple in-vivo devices. This may allow, for example, the electro-stimulation to take place under direct vision of the in-vivo imaging device 202.

In some embodiments, the length of connector 205 may be approximately 10 millimeters, or under 10 millimeters. This may allow, for example, the in-vivo imaging device 201 to acquire images of the in-vivo electro-stimulation device 202, e.g., without interference of a tissue or potion of body lumen 210 that may be sucked in to a larger space in between two devices.

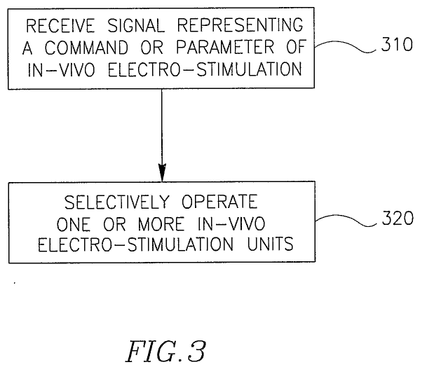

FIG. 3 is a flow-chart of a method of in-vivo electro-stimulation in accordance with some embodiments of the invention. The method may be used, for example, in conjunction with one or more components, devices and/or systems described herein, and/or other suitable in-vivo devices and/or systems.

As indicated at box 310, the method may optionally include, for example, receiving a signal representing a command or a parameter related to in-vivo electro-stimulation.

As indicated at box 320, the method may optionally include, for example, selectively operating one or more ESUs in-vivo, e.g., based on the received signal. This may be performed, for example, by selectively providing power to one or more ESUs, for example, to induce movement of an in-vivo device as a result of a contraction caused by the electro-stimulation.

Other suitable operations of sets of operations may be used.

Various aspects of the various embodiments disclosed herein are combinable with the other embodiments disclosed herein.

According to some embodiments a visual/imaging element may be combined with pace-making in the gut. For example a device according to embodiments of the invention may be used to pace the stomach, esophagus, small intestine or colon to induce a contraction for nausea, vomiting, post-operative ileus, constipation, obesity, gastro-esophageal reflux and gastroparesis. A device according to embodiments of the invention may be used to stimulate the stomach in patients with impaired gastric emptying.

According to some embodiments attaching a capsule with electrostimulation to the wall of the stomach could be used to stimulate the stomach and to visually confirm that there was a contraction. Using an in vivo imaging device according to embodiments of the invention may also assist in finding optimal settings, for example, for inducing a contraction.

According to embodiments of the invention electrostimulation may squeeze a tissue against a device (e.g., capsule). This may be used an effector mechanism. For example, tissue can be squeezed into a cavity in the device for biopsy (i.e. taking a bit of tissue).

Additionally, electrostimulation might be used to alter capillary permeability, for example, so that drugs released from a device can be “squeezed” into a target tissue or location.

For some purposes it might be helpful aspirate small amounts of fluid into a capsule for example, for micro-chemical analysis. Electrostimulation might be used to exert a force to drive tissue fluid from a visualized target into a capillary chamber in the device.

According to additional embodiments electrostimulation can be used to cause tissue to enter a cavity in a device and then a pin may be fired through the tissue which could remotely attach a visual capsule to the wall of the stomach or intestine without having to use suction. Attachment of imaging capsules to the GI tract for longer periods of observation could be valuable especially for observing bleeding.

A device, system and method in accordance with some embodiments of the invention may be used, for example, in conjunction with a device which may be inserted into a human body. However, the scope of the present invention is not limited in this regard. For example, some embodiments of the invention may be used in conjunction with a device which may be inserted into a non-human body or an animal body.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

1-18. (canceled)

19. An autonomous in-vivo device comprising:
an in-vivo electro-stimulation unit to electro-stimulate at least a portion of a body lumen,
an indicator operatively coupled to the in-vivo electro-stimulation unit, to indicate movement of said device; and
at least an imager to acquire in-vivo images.

20. The autonomous in-vivo device of claim 19, wherein the indicator indicates a direction of movement of said device.

21. The autonomous in-vivo device of claim 19, further comprising:
a receiver to receive a signal representing an in-vivo electro-stimulation command.

22. The autonomous in-vivo device of claim 19, further comprising:
a power source to selectively provide power to the in-vivo electro-stimulation unit based on a received signal.

23. The autonomous in-vivo device of claim 19, further comprising:
a power source to selectively provide power to the in-vivo electro-stimulation unit based on a pre-programmed in-vivo electro-stimulation scheme.

24. The autonomous in-vivo device of claim 19, wherein the in-vivo electro-stimulation unit is located in a non-obscuring location relative to a field-of-view imaged by the imager.

25. The autonomous in-vivo device of claim 19, wherein the indicator is a visual indicator.

26. The autonomous in-vivo device of claim 19, wherein the indicator comprises at least one illumination source.

27. The autonomous in-vivo device of claim 19, wherein the indicator provides a visual indication upon operation of the in-vivo electro-stimulation unit.

28. The autonomous in-vivo device of claim 19, wherein the in-vivo electro-stimulation unit comprises:
a front in-vivo electro-stimulation unit located at a front side of the autonomous in-vivo device; and
a back in-vivo electro-stimulation unit located at a back side of the autonomous in-vivo device.

29. The autonomous in-vivo device of claim 28, further comprising:
a first indicator operatively coupled to the front in-vivo electro-stimulation unit; and
a second indicator operatively coupled to the back in-vivo electro-stimulation unit.

30. The autonomous in-vivo device of claim 29, wherein the first indicator is to generate a visual indication having a first color, and the second indicator is to generate a visual indication having a second color, wherein the first color is different than the second color.

31. A system comprising:
an autonomous in-vivo imaging device including at least an imager to acquire in-vivo images;
an autonomous in-vivo electro-stimulation device including at least an electro-stimulation unit, and at least one indicator operatively coupled to the electro-stimulation unit to indicate movement of the system; and
a connector to connect the in-vivo imaging device and the in-vivo electro-stimulation device.

32. The system of claim 31, wherein the indicator indicates a direction of movement of the system.

33. The system of claim 31, wherein the indicator is a visual indicator.

34. The system of claim 31, wherein the imager is to acquire an in-vivo image including at least a portion of the indicator.

35. A method comprising:
receiving a signal representing an in-vivo electro-stimulation command;
based on the in-vivo electro-stimulation command, operating an in vivo device comprising an in-vivo electro-stimulation unit; and
indicating movement of the in-vivo device.

36. The method of claim 35, wherein the step of indicating movement comprises indicating a direction of movement of the in vivo device.

37. The method of claim 35, comprising:
acquiring in-vivo images.

38. The method of claim 35, comprising:
transmitting image data.

39. The method of claim 35, comprising:
selectively activating or de-activating the in-vivo electro-stimulation unit.
